(19) United States
(12) Patent Application Publication
(10) Pub. No.: US 2026/0325647 A1
(43) Pub. Date: Oct. 22, 2026

(54) UNIVERSAL ANTIGEN PRESENTING CELLS AND USES THEREOF

(71) Applicant: BOARD OF REGENTS, THE UNIVERSITY OF TEXAS SYSTEM, Austin, TX (US)

(72) Inventors: Sonny O. T. ANG, Houston, TX (US); Enli LIU, Houston, TX (US); Elizabeth SHPALL, Houston, TX (US); Katy REZVANI, Houston, TX (US)

(73) Assignee: BOARD OF REGENTS, THE UNIVERSITY OF TEXAS SYSTEM, Austin, TX (US)

(21) Appl. No.: 19/734,174

(22) Filed: Jul. 8, 2026

Related U.S. Application Data
(63) Continuation of application No. 16/970,930, filed on Feb. 21, 2019.
(60) Provisional application No. 62/633,587, filed on Feb. 21, 2018.

(51) Int. Cl. A61K 45/06; A61K 39/00; A61K 39/395; A61K 40/15; A61K 40/31; A61K 40/41; C07K 14/54; C07K 14/705; C07K 16/28; C12N 5/0783; C12N 15/86

(52) U.S. Cl. CPC A61K 45/06; A61K 39/39541; A61K 40/15; A61K 40/31; A61K 40/418; C07K 14/54; C07K 14/70503; C07K 14/70575; C07K 16/2887; C12N 5/0636; C12N 5/0646; C12N 15/86; A61K 2039/505; A61K 2239/48; C12N 2500/90; C12N 2501/2302; C12N 2502/30; C12N 2740/10043

(57) ABSTRACT

Provided herein are universal antigen presenting cells. Also provided herein are methods of expanding immune cells using the UAPCs and methods for the treatment of a disease, such as cancer, using the expanded immune cells.

This application is a continuation of U.S. patent application Ser. No. 16/970,930 filed Aug. 18, 2020, which is a national phase application under 35 U.S.C. § 371 that claims priority to International Application No. PCT/US2019/018989 filed Feb. 21, 2019, which claims the benefit of U.S. Provisional Application No. 62/633,587 filed Feb. 21, 2018, all of which are incorporated herein by reference in their entirety.

### INCORPORATION OF SEQUENCE LISTING

The instant application contains a Sequence Listing which has been submitted in XML format and is hereby incorporated by reference in its entirety. Said XML copy, created on Jul. 7, 2026, is named MDACP1182USC1-Sequence-Listing.xml and is 19,700 bytes in size.

### BACKGROUND

### 1. Field

The present invention relates generally to the fields of medicine and immunology. More particularly, it concerns antigen presenting cells and uses thereof, such as expanding natural killer (NK) cells.

### 2. Description of Related Art

Natural killer (NK) cells do not need previous exposure to antigens for activation which is in contrast to T cells, making them highly effective in immuno-surveillance against tumors. While prior activation is not required, the immense killing powers of NK cells need stringent controls to forestall unintended cytotoxicity and autoimmunity. Generation of clinically sufficient quantities and optimally functional NK cells is another impediment. Thus, there is a need to focus on the molecular machinery governing NK cell biology to discriminate “resistant” from “sensitive” target cells to design helper cells to enhance NK-cell mediated cancer immunotherapy.

### SUMMARY

Accordingly, certain embodiments of the present disclosure provide methods and compositions concerning the manufacture, expansion, quality control, and functional characterization of clinical-grade NK cells intended for cell and immunotherapy.

In a first embodiment, there is provided a universal antigen presenting cell (UAPC) engineered to express (1) CD48 and/or CS1 (CD319), (2) membrane-bound interleukin-21 (mbIL-21), and (3) 41BB ligand (41BBL). In some aspects, the UAPC expresses CD48. In other aspects, the UAPC expresses CS1. In particular aspects, the UAPC expresses CD48 and CS1.

In some aspects, the UAPC has essentially no expression of endogenous HLA class I, II, or CD1d molecules. In certain aspects, the UAPC expresses ICAM-1 (CD54) and LFA-3 (CD58).

In certain aspects, the UAPC is further defined as a leukemia cell-derived aAPC. In some aspects, the leukemia-cell derived UAPC is further defined as a K562 cell.

In some aspects, engineered is further defined as retroviral transduction. In some aspects, the retroviral transduction is further defined as transduction of a viral construct of SEQ ID NO: 1 and/or SEQ ID NO:2. In certain aspects, the UAPC is irradiated.

In a further embodiment, there is provided a method for expanding immune cells comprising culturing the immune cells in the presence of an effective amount of UAPCs of the embodiments (e.g., a universal antigen presenting cell (UAPC) engineered to express (1) CD48 and/or CS1 (CD319), (2) membrane-bound interleukin-21 (mbIL-21), and (3) 41BB ligand (41BBL)). In some aspects, the immune cells and UAPCs are cultured at a ratio of 3:1 to 1:3, such as 3:1, 3:2, 1:1, 1:2, or 1:3. In particular aspects, the immune cells and UAPCs are cultured at a ratio of 1:2.

In some aspects, the expanding is in the presence of IL-2. In specific aspects, the IL-2 is present at a concentration of 10-500 U/mL, such as 10-25, 25-50, 50-75, 75-10, 100-150, 150-200, 200-250, 250-300, 300-350, 350-400, or 400-500 U/mL. In certain aspects, the IL-2 is present at a concentration of 100-300 U/mL. In particular aspects, the IL-2 is present at a concentration of 200 U/mL. In some aspects, the IL-2 is recombinant human IL-2. In specific aspects, the IL-2 is replenished every 2-3 days, such as every 2 days or 3 days.

In certain aspects, the UAPCs are added at least a second time. In some aspects, the immune cells are NK cells or T cells. In particular aspects, the immune cells are NK cells.

In particular aspects, the immune cells are derived from cord blood (CB), peripheral blood (PB), stem cells, or bone marrow. In specific aspects, the stem cells are induced pluripotent stem cells. In some aspects, the immune cells are obtained from CB. In particular aspects, the CB is pooled from 2 or more individual cord blood units. In specific aspects, the CB is pooled from 3, 4, 5, 6, 7, or 8 individual cord blood units.

In some aspects, the NK cells are CB mononuclear cells (CBMCs). In certain aspects, the NK cells are further defined as CD56+NK cells.

In certain aspects, the method is performed in serum-free media.

In additional aspects, the immune cells are engineered to express a chimeric antigen receptor (CAR). In some aspects, the CAR comprises a CD19, CD123, mesothelin, CD5, CD47, CLL-1, CD33, CD99, U5snRNP200, CD200, CS1, BAFF-R, ROR-1, or BCMA antigen-binding domain. In some aspects, the CAR is a humanized CAR. In some aspects, the CAR is co-expressed with IL-15. In certain aspects, the CAR is co-expressed with a suicide gene. In some aspects, the suicide gene is CD20, CD52, EGFRv3, or inducible caspase 9.

Further provided herein is a population of expanded immune cells produced according to the embodiments (e.g., culturing the immune cells in the presence of an effective amount of UAPCs of the embodiments (e.g., a universal antigen presenting cell (UAPC) engineered to express (1) CD48 and/or CS1 (CD319), (2) membrane-bound interleukin-21 (mbIL-21), and (3) 41BB ligand (41BBL)).

In another embodiment, there is provided a pharmaceutical composition comprising the population of expanded immune cells of the embodiments and a pharmaceutically acceptable carrier. Further provided herein is a composition comprising an effective amount of the expanded immune cells of the embodiments for use in the treatment of a disease or disorder in a subject.

A further embodiment provides a method of treating a disease or disorder in a subject comprising administering a therapeutically effective amount of the expanded immune cells of the embodiments to the subject.

In some aspects, the disease or disorder is cancer, inflammation, graft versus host disease, transplant rejection, an autoimmune disorder, an immunodeficiency disease, a B cell malignancy, or an infection. In particular aspects, the cancer is a leukemia. In some aspects, the leukemia is an acute lymphoblastic leukemia (ALL), chronic lymphocytic leukemia (CLL), acute myelogenous leukemia (AML), or a chronic myelogenous leukemia (CML). In particular aspects, the disorder is graft versus host disease (GVHD). In some aspects, the disorder is multiple sclerosis, inflammatory bowel disease, rheumatoid arthritis, type I diabetes, systemic lupus erythrematosus, contact hypersensitivity, asthma or Sjogren's syndrome. In some aspects, the subject is a human.

In certain aspects, the immune cells are allogeneic. In some aspects, the immune cells are autologous. In some aspects, the immune cells are NK cells or T cells.

In additional aspects, the method further comprises administering at least a second therapeutic agent. In some aspects, the at least a second therapeutic agent is a therapeutically effective amount of an anti-cancer agent, immunomodulatory agent, or an immunosuppressive agent. In certain aspects, the anti-cancer agent is chemotherapy, radiotherapy, gene therapy, surgery, hormonal therapy, anti-angiogenic therapy or immunotherapy.

In some aspects, the immunosuppressive agent is a calcineurin inhibitor, an mTOR inhibitor, an antibody, a chemotherapeutic agent irradiation, a chemokine, an interleukins or an inhibitor of a chemokine or an interleukin.

In further aspects, the immune cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion. In some aspects, the second therapeutic agent is an antibody. In certain aspects, the antibody if a monoclonal, bispecific, or trispecific antibody. In some aspects, the antibody is rituximab.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIG. 1: Zipper model of human natural killer cell receptors and target cell ligand interactions. Human NK cells interact with target cells via many receptors, including killer immunoglobulin receptors (KIRs), natural cytotoxicity receptors (NCRs), NKG2 family of receptors, nectin binding receptors, SLAM family receptors and others. Receptors on NK cells are activating (KIR2DL4, KIR3DS1, NKp30, NKp44, NKp46, NKp80, CD94-NKG2C, DAP10, NKG2D, DAP10, CRTAM, DNAM, 2B4, NTB-A, CD3 zeta, CD100, CD160) or inhibitory (KIR2DL1/2/3/5A/5B, KIR3DL1/2/3, CD94-NKG2A, TIGIT, CD96, CEACAM-1, ILT2/LILRB1, KLRG1, LAIR1, CD161, Siglec-3/7/9)

FIGS. 2A-2E: (FIG. 2A-2B) Fold expansion of fresh or frozen NK cells with IL-2 or APCs. (FIG. 2C) Flow cytometry at Day 0, Day 7, or Day 14 for expression of CD3 and CD56 in NK cells. (FIG. 2D) Cell number of NT-NK or SG4-NK cells stimulated with APCs. (FIG. 2E) Growth kinetics of NK cells stimulated with Clone 9 APC or UAPC.

FIGS. 3A-3D: (FIG. 3A) Flow cytometry of parental K562 cells for indicated markers. No observed expression of mb-IL21, 41BBL, CD48, and SLAMF7 (CS1). (FIG. 3B) Flow cytometry analysis of APCs post-transduction of clone 46 (mbIL-21, 41BBL). (FIG. 3C) Flow cytometry analysis of APCs post-transduction of uAPC construct for expression of CD48 (nmIL-21, 41BBL, and CD48). (FIG. 3D) Flow cytometry analysis of APCs post-transduction of uAPC2 construct for expression of CS1 (mbIL-21, 41BBL, and CS1).

FIGS. 4A-4D: MMLV-retroviral transfer construct maps and annotations. (FIG. 4A) Retroviral transfer vectors for mb-IL21. (FIG. 4B) Retroviral transfer vectors for 41BBL. (FIG. 4C) Retroviral transfer vectors for CD48-Katushka. (FIG. 4D) Retroviral transfer vectors for CS1-EGFP.

### DESCRIPTION OF ILLUSTRATIVE EMBODIMENTS

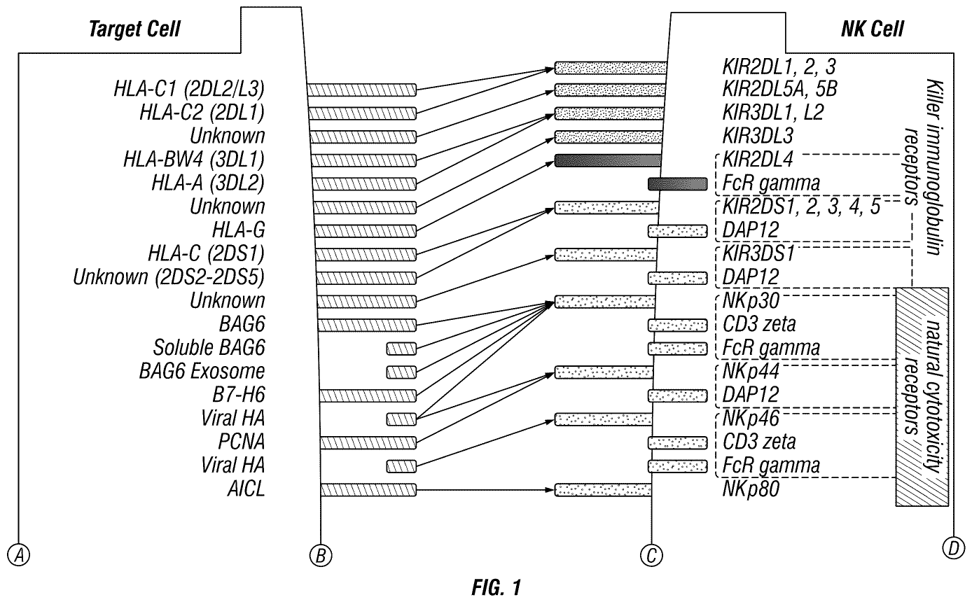

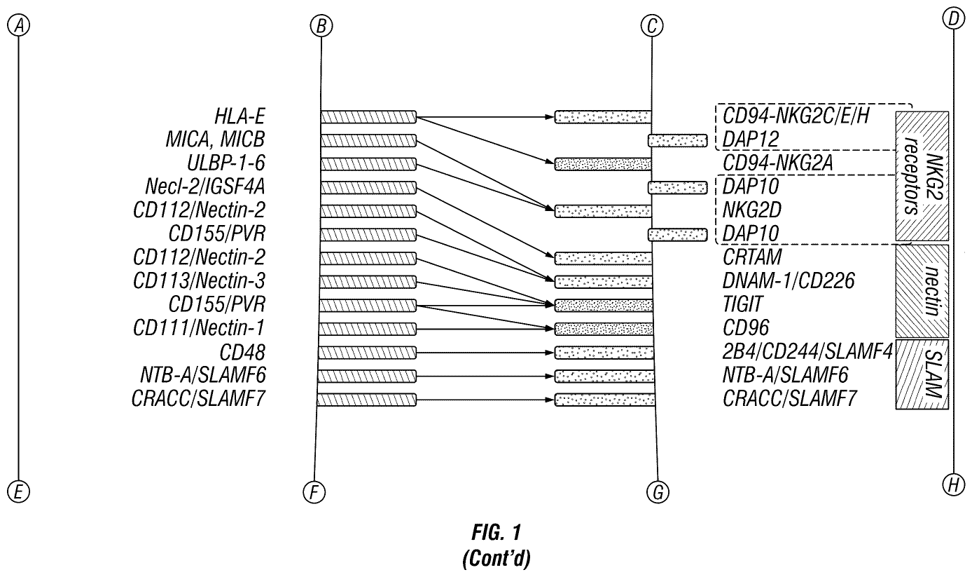

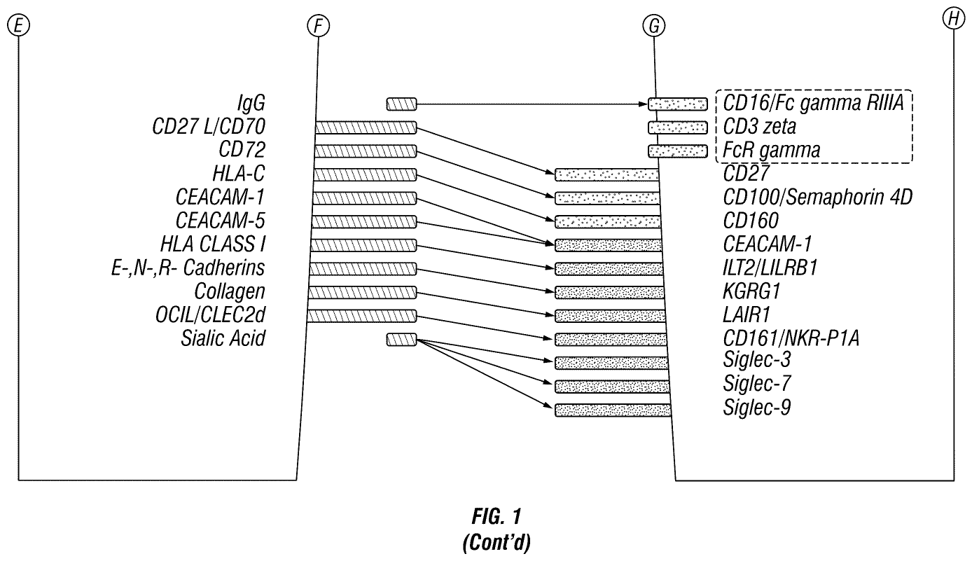

In the present studies, NK cells were initially characterized for their anti-tumoral and anti-allogeneic cytolytic functions, distinct from other components of the innate immunity compartment. A myriad of immunoreceptors governing the behaviors of NK cells are illustrated as a “zipper” graph in FIG. 1. The array of signal transducers can be broadly categorized into 2 large groups, namely activating versus inhibitory. Structural and signaling modalities further fractionate the categories into related families of immunoreceptors. Although the membrane proteome of NK cells has yet to be fully elucidated, the functional redundancies within the NK cell “zipper” were leveraged to modulate cytolytic functions while preserving the complex and dynamic balance between complementary and antagonistic pathways.

Accordingly, certain embodiments of the present disclosure provide methods and compositions concerning the manufacture, expansion, quality control, and functional characterization of clinical-grade NK cells intended for cell and immunotherapy. Growing and molding clinically relevant numbers of NK cells for infusion into patients while meeting time constraints are challenging even in the best of circumstances. In certain aspects, the disclosed methods and compositions detail the technical processes of NK cell manufacture, details and kinetics of achievable NK cell expansions, and molecular characterization to verify successful cellular molding.

In further embodiments, there is provided herein a robust platform technology embodied as universal antigen presenting cells (UAPC) to condition, mold, and weaponize human natural killer (NK) cells against tumors. “UAPC(s)” refer herein to antigen presenting cells designed for the optimized expansion of immune cells, such as NK cells. The present UAPCs were generated by a unique combination of co-stimulatory molecules to overcome inhibitory signals and induce optimal and specific NK cell killing function. Extensive testing showed UAPCs fine tunes NK cellular machinery and improved tumor elimination. The present UAPCs may be used for NK cell-mediated cancer immunotherapy.

Exemplary APCs are generated by enforced expression of membrane-bound interleukin 21 (mbIL-21) and 4-1BB ligand in the NK cell-sensitive K562 antigen-presenting cell line (APC) (referred to as clone 46). In another embodiment, UAPCs were produced by enforced expression of mbIL-21, 4-1BB ligand, and CD48 in K562 cells (termed universal APC (UAPC)). In another embodiment, UAPCs were generated by enforced expression of mbIL-21, 4-1BB ligand, and CS1 in K562 cells (termed UAPC2). The UAPCs may be generated to express mbIL-21, 41BBL, and an NK-cell specific antigen, such as a SLAM family antigen (Table 1).

The UAPC platform may also be applied to expand other immune effectors including T cells (e.g., alpha-beta and gamma-delta T cells). The immune cells, such as NK cells and T cells may be derived from peripheral blood, umbilical cord blood, bone marrow or stem cells (including induced pluripotent stem cells). These immune cells may be subjected to ex vivo expansion and activation using the present UAPCs, which is required for cultivation to reach meaningful quantities for clinically relevant applications such as cancer immunotherapy.

Thus, the present disclosure provides a series of helper cell lines designed and manufactured to condition, modulate, prime and expand human immune, such as NK, cells for cancer immunotherapy.

### II. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B cell or a T cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “haplotyping or tissue typing” refers to a method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are six genetic loci important in transplantation, designated HLA-A, HLA-B, HLA-C, and HLA-DR, HLA-DP and HLA-DQ. At each locus, there can be any of several different alleles.

A widely used method for haplotyping uses the polymerase chain reaction (PCR) to compare the DNA of the subject, with known segments of the genes encoding MHC antigens. The variability of these regions of the genes determines the tissue type or haplotype of the subject. Serologic methods are also used to detect serologically defined antigens on the surfaces of cells. HLA-A, -B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The pattern of cells destroyed by lysis indicates the degree of histologic incompatibility. If, for example, the lymphocytes from a person being tested for HLA-A3 are destroyed in a well containing antisera for HLA-A3, the test is positive for this antigen group.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B cells, endothelial cells, activated T cells, and dendritic cells, or molecules, naturally occurring or synthetic capable of presenting antigen, such as purified MHC Class I molecules complexed to ÿ2-microglobulin.

### III. Universal Antigen Presenting Cells

Some embodiments of the present disclosure concern the production and use of universal antigen present cells (UAPCS). The UAPCs may be used for the expansion of immune cells, such as NK cells and T cells. The UAPCs may be engineered to express membrane-bound IL-21 (mbIL-21) and 41BBL (CD137 ligand).

The UAPCs may be engineered to express CD137 ligand and/or a membrane-bound cytokine. The membrane-bound cytokine may be mIL-21 or mIL-15. In particular embodiments, the UAPCs are engineered to express CD137 ligand and mIL-21. The APCs may be derived from cancer cells, such as leukemia cells. The APCs may not have any expression of endogenous HLA class I, II, or CD1d molecules. The APCs may express ICAM-1 (CD54) and LFA-3 (CD58). In particular, the APCs may be K562 cells, such as K562 cells engineered to express CD137 ligand and mIL-21. The APCs may be irradiated. The engineering may be by any method known in the art, such as retroviral transduction.

Cytokines exert very potent controls over entire classes of immune cells (including NK cells), affecting cellular fate, activity and efficacy of cellular responses. The power of cytokine stimulation to activate NK cells confirms their “natural” effector functions are highly susceptible to environmental intervention, and that priming may regulate NK cell behaviors in vivo.

To address the issue of acquiring clinically relevant quantities of NK cells, the present methods may use interleukin (IL-21) as the driver for NK cell expansion in the present UAPC platform technology. In humans, NK-cell stimulation with IL-10 and IL-21 induces NKG2D expression in a STAT3-dependent manner. While the cytokine receptors share similar cellular signaling components in many immune cells, NK cell-specific signaling is dependent on the IL-21 receptor-STAT3 nexus for proliferation.

Cytokine signaling is important for maintenance of lymphocyte survival and proliferation. In vivo, IL-2 administration is the only FDA approved method for expanding NK cells. IL-15, another potent NK cell activator, is undergoing Phase I clinical trial as a possible alternative to IL-2, but may have significant toxicity following systemic administration. Apart from significant toxicities, these cytokines also induce proliferation of T cells while limiting the persistence of NK cells. Despite sharing the same receptor for signal transduction, IL-2, IL-4, IL-7, IL-9, IL-15, and IL-21 receptors have distinct and specific effects on diverse cells.

### B. Membrane-Bound IL-21

In certain embodiments, for specifically energizing NK cells, IL-21 may be used for producing the present UAPCs. IL-21 receptor (IL21R), a close relation to the IL-2 receptor beta chain capable of transducing signals through its dimerization with the common cytokine receptor gamma chain (gamma (c), is upregulated in activated human NK cells (cells ready to be triggered). While IL-21, a type I cytokine, can modulate T, B, and NK cell functions, the present studies found that only human NK cells experience significant expansion via activation of the IL21 receptor signaling pathway (i.e., 1000-fold over 21 days). Conversely, IL21 plays an important role in the contraction of CD8+ T cells.

IL21R signaling is powered mainly by STAT3, a highly potent cellular proliferation activator. The IL21R-STAT3 nexus is driven by IL-21-induced STAT3 DNA binding to GAS and cis-inducible elements as verified by immunoprecipitation and Western blotting with anti-phosphotyrosine antibody. The molecular basis of IL21 mediated proliferation can be specifically traced to tyrosine 510 (Y510) on IL21R, which mediates IL-21-induced phosphorylation of STAT1 and STAT33. This mechanism is underscored by dampened IL-21 responses in Stat1/Stat3 double knock-out mice.

IL21R signaling is important for NK cytotoxicity. Human IL21R deficiency is linked to impaired cytolysis of 51Cr-labeled K562 target cells, while antibody-dependent cellular cytotoxicity is unaffected. The monogenic non-embryonic lethal defect (loss-of-function mutation in IL21R) presents an excellent opportunity delineate, and thus useful in modeling, enhancing, and shaping innate NK cytolytic responses.

IL-21 signaling selectively molds NK cell subsets, even though both CD56dim and CD56bright cell populations harbor similar numbers of surface IL21R. IL-21 induction of STAT1 and STAT3 phosphorylation is higher in CD56bright vs CD56dim NK cells. In contrast, IL-21 has no effect on STAT5 activation, an IL-2 activation pathway which also drives T cell expansion. In addition to STAT3 activation, IL-21 signaling also involves the MAPK, and PI3K pathways and induces expression of innate immune responsive genes including IFN-gamma, T-bet, IL-12Rbeta2, and IL-18R in NK cells, priming them to kill tumor cells.

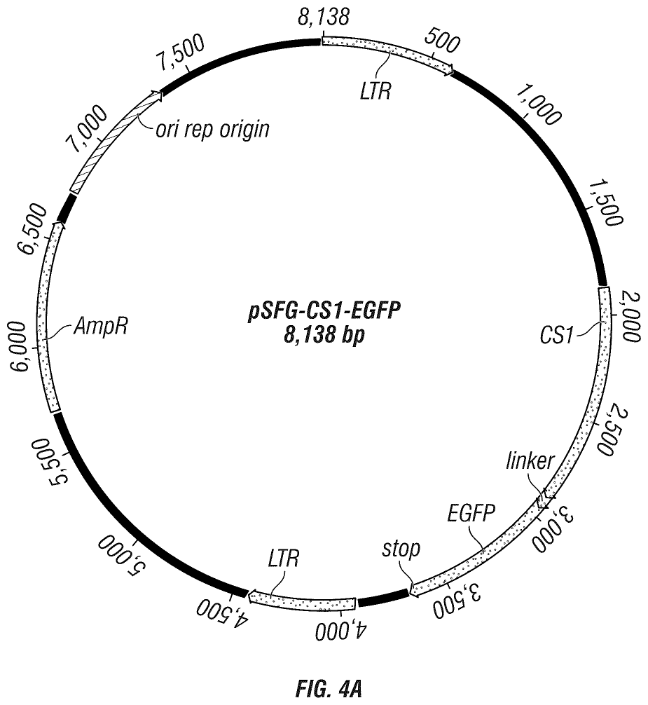

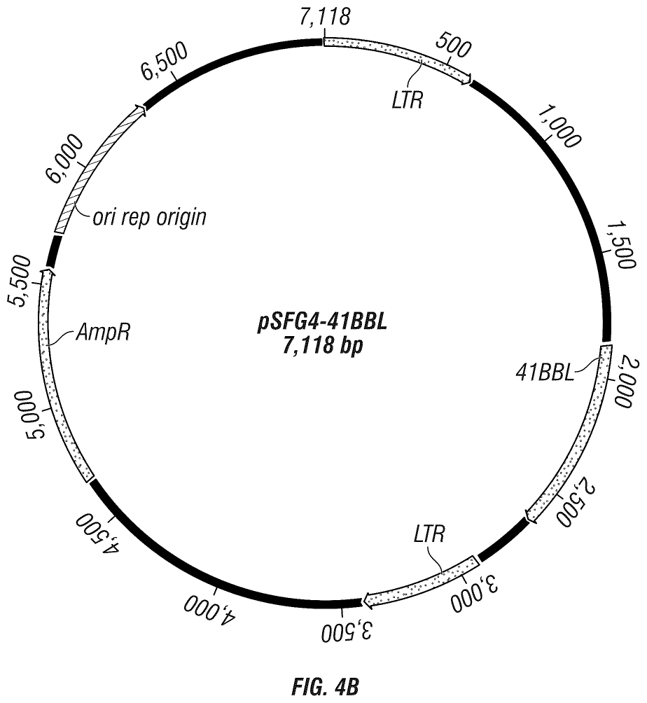

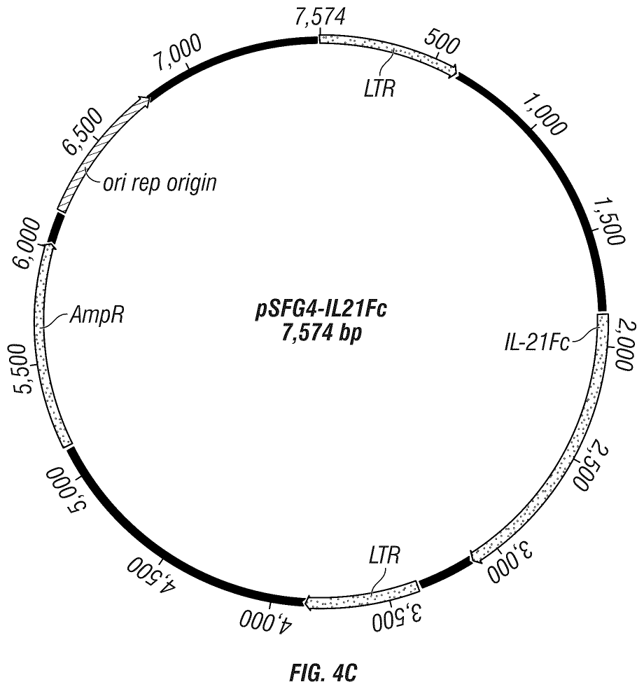

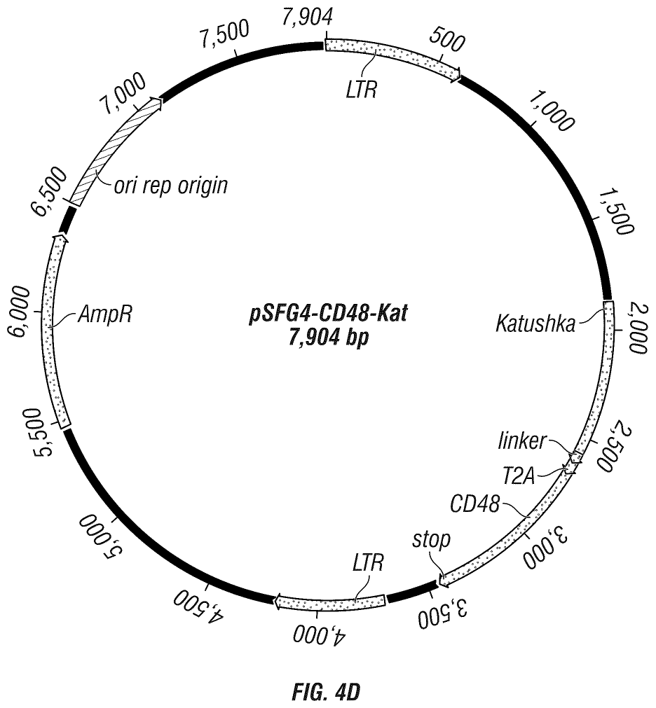

For efficient utilization of IL-21, expression of mbIL-21 (FIG. 4A) may be used to concentrate and localize trans interaction with IL21R on NK cells. The membrane proximity of mbIL21 ensures ready availability where it is needed, thus, it can sustain optimal cell proliferation without large quantities and concentrations of exogenously supplied IL-21. Co-cultures with irradiated K562-mb15-41BBL induced a median 21.6-fold expansion of CD56+CD3-NK cells from peripheral blood. This expansion is higher compared to stimulation with just soluble cytokines from the shared γc family including IL-2, IL-12, IL-15, IL-21 alone or in combinations. By comparison, the present UAPCS can expand NK cells by at least 1000-fold (3-log) over 14-21 days. Expression of mbIL21 on UAPCs can also obviate the need for exogenous clinical-grade cytokine.

### C. 4-1BBL (4-1BB Ligand, CD137 Ligand, CD137L, TNFSF9)

In addition to the notion of cytokine priming of NK cell function, direct physical interactions with activating molecules in NK cells result in enhanced cellular proliferative responses. CD137 (4-1BB) is a member of the tumor necrosis receptor (TNF-R) gene family, which mediates cell proliferation, differentiation, and programmed cell death (apoptosis). The murine receptor was first characterized followed by the human homolog, which shares a 60% identity at the amino acid level, with significant conservation in the cytoplasmic/signaling domain. CD137 is mainly expressed in activated T-cells and NK cells, with varying levels detectable in thymocytes, myeloid cells, and endothelial cells at sites of inflammations. Physiological CD137 signaling is mediated via 1) NF-κB which promotes survival through Bcl-XL activation and 2) PI3K/ERK1/2 pathway which specifically drive cell cycle progression.

In activated NK cells, CD137 is a cytokine-inducible costimulatory molecule, which in turn drives anti-tumor responses in NK cells by increasing cellular proliferation and IFN-γ secretion. Studies using CD137L−/−knockout mice elucidated the importance of CD137/CD137L signaling in developing anti-tumor immune cells. CD137−/−knockout mice had a 4-fold higher frequency of tumor metastases compared to control mice.

CD137 ligand (CD137L, 4-1BB ligand), a 34 kDa glycoprotein member of the TNF superfamily, is detected mainly on activated antigen-presenting cells (APC), including B cells, macrophages, and dendritic cells as well as transiently expressed at low levels on activated T cells. Human CD137L is only 36% homologous compared to the murine counterpart. In line with anti-tumor efficacies of agonistic CD137 antibodies, CD137L binding has been shown to elicit CTL and anti-tumor activities.

Accordingly, the present UAPCS may be engineered to express 4-1BB ligand, the physiological counter-receptor for CD137 for stimulation.

### D. SLAM/CD48

Apart from cytokine conditioning and 4-1BBL co-stimulation, direct physical interactions between NK and target cells also influence the cellular responses i.e. killing of the target cells. The signaling lymphocytic activating molecule (SLAM, previously also known as CD2 superfamily) family of homologous immunoglobulin receptors, which are widely expressed and play critical roles in the immune system, are especially important in terms of intercellular interactions.

Accordingly, the UAPCs of the present disclosure may express one or more SLAM family antigens. SLAM family members include CD2, CD48, CD58 (LFA-3), CD244 (2B4), CD229 (Ly9), CD319 (CS1 (CD2 subset 1); CRACC (CD2-like receptor activating cytotoxic cells)), and CD352 (NTB-A (NK-T-B antigen)). In particular aspects, the UAPCs express CD48 and/or CS1. NK cells express at least three members of the SLAM family (SLAMF). They are 2B4, NK, T- and B-cell antigen (NTB-A), and CD2-like receptor-activating cytotoxic cells (CRACC), which recognize their respective ligands CD48, NTB-A, and CRACC on target cells and possibly on other NK cells. While SLAMF1, 3, 5, 6, 7, 8, and 9 are homophilic (self-ligand) receptors, SLAMF2 and SLAMF4 are counter receptors (heterophilic) to each other. The broad range (three orders of magnitude) in known homophilic affinities (dissociation constant, Kd, of <1 μM to 200 μM) points to a mechanistic basis for the overlapping, but distinct, signaling mechanisms for the SLAM glycoproteins.

TABLE 1
SLAM family members binding affinities.

| | | | | | Counter | |
|---|---|---|---|---|---|---|
| SLAMF | CD | Name | Aliases | Interaction | Receptor | Affinity |
| 1 | 150 | SLAM | SLAMF1, | homophilic | | 200 |
| | | | CD150, | | | |
| | | | CDw150 | | | |
| 2 | 48 | CD48 | BCM1, | heterophilic | CD2 (rodents) | >500 |
| | | | BLAST, | | | |
| | | | BLAST1, | | | |
| | | | MEM-102 | | | |
| | | | | heterophilic | 2B4 | 8 |
| 3 | 229 | LY9 | hly9, | homophilic | | |
| | | | mlymphocyte | | | |
| | | | antigen 9 | | | |
| 4 | 244 | 2B4 | NAIL, | heterophilic | CD48 | 8 |
| | | | NKR2B4, | | | |
| | | | Nmrk | | | |
| 5 | 84 | CD84 | LY9B | homophilic | | <1 |
| 6 | 352 | NTB-A | KALI, KALIb, | homophilic | | 2 |
| | | | Ly108, | | | |
| | | | NTBA, | | | |
| | | | SF2000 | | | |
| 7 | 319 | CRACC | CS1, 19A | homophilic | | |
| 8 | 353 | | BLAME, | homophilic | | |
| | | | SBBI42 | | | |
| 9 | | | CD2F10, | homophilic | | |
| | | | CD84H1, | | | |
| | | | SF2001, | | | |
| | | | CD2F-10, | | | |
| | | | CD84-H1 | | | |

In particular embodiments, the present UAPCs are engineered to express CD48 to bolster cell-to-cell interaction to enhance NK cellular responses. CD48 is a glycosylphosphatidylinositol-anchored protein (GPI-AP) found on the surface of NK cells, T cells, monocytes, and basophils, and participates in adhesion and activation pathways in these cells. Despite its lack of an intracellular domain, stimulation of CD48 induces rearrangement of signaling factors in lipid rafts, Lck-kinase activity, and tyrosine phosphorylation. As an adhesion and co-stimulatory molecule, CD48 induces numerous effects in B and T lymphocytes, NK cells, mast cells, and eosinophils. In human NK cells, CD48 is the counter receptor for 2B426, an important activator of NK cells. The heterophilic interaction is thought to compete for CD244 interaction with MHC-I. 2B4/CD48 interaction thus induces activation signals in human NK cells, whereas in murine NK cells it sends inhibitory signals.

While 2B4-CD48 interactions between cells of the same population, i.e. NK cell-NK cell interactions or T cell-T cell interactions, leads to enhanced activation, by expressing CD48 on APCs, such as K562 myeloid cell line which is normally devoid of CD48, the UAPCs can turn on the potent 2B4 signaling pathway on NK cells in trans.

### E. SLAM/CS1

In some embodiments, the present UAPCs are engineered to express CS1, another member of the SLAM family, as a co-stimulatory molecule to switch on the killing powers of NK cells. Unlike CD48 which counter binds to 2B4, CS1 interactions are homophilic, and can be characterized in the context of cis vs trans interactions. K562 cells which are normally free of CS1 may be engineered to express CS1.

### F. Delivery of Nucleic Acids

The mbIL-21, 41BBL, and co-stimulatory antigen may be engineered by any of the methods known in the art. The nucleic acid typically is administered in the form of an expression vector, such as a viral expression vector. In some aspects, the expression vector is a retroviral expression vector, an adenoviral expression vector, a DNA plasmid expression vector, or an AAV expression vector. In some aspects, the delivery is by delivery of one or more vectors, one or more transcripts thereof, and/or one or more proteins transcribed therefrom, is delivered to the cell.

Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g. retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

### IV. Immune Cells

Some embodiments of the present disclosure concern the isolation and expansion of immune cells, such as a NK cells or T cells, such as for cancer immunotherapy.

In certain embodiments, immune cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the immune cells may be isolated from cord blood (CB), peripheral blood (PB), bone marrow, or stem cells. In particular embodiments, the immune cells are isolated from pooled CB. The CB may be pooled from 2, 3, 4, 5, 6, 7, 8, 10, or more units. The immune cells may be autologous or allogeneic. The isolated immune cells may be haplotype matched for the subject to be administered the cell therapy. NK cells can be detected by specific surface markers, such as CD16, CD56, and CD8 in humans.

In certain aspects, the NK cells are isolated by the previously described method of ex vivo expansion of NK cells (Spanholtz et al., 2011; Shah et al., 2013). In this method, CB mononuclear cells are isolated by ficoll density gradient centrifugation. The cell culture may be depleted of any cells expressing CD3 and may be characterized to determine the percentage of CD56+/CD3-cells or NK cells. In other methods, umbilical CB is used to derive NK cells by the isolation of CD34+ cells. The method may comprise depletion of CD3, CD14, and/or CD19-positive cells.

The isolated immune cells may be expanded in the presence of the present UAPCs. The expansion may be for about 2-30 days, such as 3-20 days, particularly 12-16 days, such as 12, 13, 14, 15, 16, 17, 18, or 19 days, specifically about 14 days. The immune cells and UAPCS may be present at a ratio of about 3:1-1:3, such as 2:1, 1:1, 1:2, specifically about 1:2. The expansion culture may further comprise cytokines to promote expansion, such as IL-2. The IL-2 may be present at a concentration of about 10-500 U/mL, such as 100-300 U/mL, particularly about 200 U/mL. The IL-2 may be replenished in the expansion culture, such as every 2-3 days. The UAPCs may be added to the culture at least a second time, such as at about 7 days of expansion.

Following expansion, the immune cells may be immediately infused or may be stored, such as by cryopreservation. In certain aspects, the cells may be propagated for days, weeks, or months ex vivo as a bulk population within about 1, 2, 3, 4, 5 days.

Expanded NK cells can secrete type I cytokines, such as interferon-y, tumor necrosis factor-a and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

### V. Chimeric Antigen Receptors

In certain embodiments, the present immune cells, such as T cells or NK cells, are genetically modified to express a chimeric antigen receptor (CAR). In some embodiments, the CAR comprises: a) an intracellular signaling domain, b) a transmembrane domain, and c) an extracellular domain comprising an antigen binding region.

A CAR recognizes cell-surface tumor-associated antigen independent of human leukocyte antigen (HLA) and employs one or more signaling molecules to activate genetically modified NK cells for killing, proliferation, and cytokine production. In certain embodiments, the present NK cells may be genetically modified by methods comprising (i) non-viral gene transfer using an electroporation device (e.g., a nucleofector), (ii) CARs that signal through endodomains (e.g., CD28/CD3-2, CD137/CD3-2, or other combinations), (iii) CARs with variable lengths of extracellular domains connecting the antigen-recognition domain to the cell surface, and, in some cases, (iv) artificial antigen presenting cells (aAPCs) derived from K562 to be able to robustly and numerically expand CAR+NK cells (Singh et al., 2011).

Embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the present methods use a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One of the Fc domains, e.g., either the CH2 or CH3 domain from human immunoglobulin may be used. The hinge, CH2 and CH3 region of a human immunoglobulin that has been modified to improve dimerization may be used. In other aspects, just the hinge portion of an immunoglobulin or portions of CD8a may be used.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

The intracellular signaling domain of a chimeric antigen receptor is responsible for activation of at least one of the normal effector functions of the immune cell in which the chimeric antigen receptor has been placed. The effector function is a specialized function of a differentiated cell, such as a NK cell. In specific embodiments, intracellular receptor signaling domains in the CAR include those of the T cell antigen receptor complex, such as the zeta chain of CD3, also Fcγ RIII costimulatory signaling domains, CD28, CD27, DAP10, CD137, OX40, CD2, alone or in a series with CD3zeta, for example. In specific embodiments, the intracellular domain (which may be referred to as the cytoplasmic domain) comprises part or all of one or more of TCR zeta chain, CD28, CD27, OX40/CD134, 4-1BB/CD137, FcεRIγ, ICOS/CD278, IL-2Rbeta/CD122, IL-2Ralpha/CD132, DAP10, DAP12, and CD40. Any part of the endogenous T cell receptor complex may be used in the intracellular domain. One or multiple cytoplasmic domains may be employed, as so-called third generation CARs have at least two or three signaling domains fused together for additive or synergistic effect, for example.

In certain embodiments of the CAR, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. The tumor antigen-binding domain may be, but is not limited to, CD19, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, EGFRvIII, or VEGFR2. The CAR may comprise a humanized scFv, such as humanized CD19 or CD123.

In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into NK cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into NK cells. Suitable vectors for use in accordance with the method of the present invention are non-replicating in the NK cells. A large number of vectors are known that are based on viruses, where the copy number of the virus maintained in the cell is low enough to maintain the viability of the cell, such as, for example, vectors based on HIV, SV40, EBV, HSV, or BPV.

The CAR may be co-expressed with a suicide gene, such as CD20, CD52, EGFRv3, or inducible caspase 9.

The CAR may comprise a tumor antigen-binding domain. The tumor antigen-binding domain may be, but is not limited to, CD19, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, EGFRvIII, or VEGFR2. The CAR may comprise a humanized scFv, such as humanized CD19, CD123, mesothelin, CD5, CD47, CLL-1, CD33, CD99, U5snRNP200, CD200, CS1, BAFF-R, ROR-1, or BCMA.

### VI. Methods of Use

Embodiments of the present disclosure concern methods for the use of the immune, such as NK or T, cells provided herein (e.g., expanded by the present UAPCs) for treating or preventing a medical disease or disorder by transfer of an immune cell population that elicits an immune response. The method includes administering to the subject a therapeutically effective amount of the expanded immune cells, thereby treating or preventing the disorder in the subject. In certain embodiments of the present disclosure, cancer or infection is treated by transfer of an immune cell population that elicits an immune response. Due to their release of pro-inflammatory cytokines, immune cells may reverse the anti-inflammatory tumor microenvironment and increase adaptive immune responses by promoting differentiation, activation, and/or recruitment of accessory immune cell to sites of malignancy.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, m malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive build up of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets.

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called acute nonlymphocytic leukemia. Chronic myelogenous leukemia (CML) occurs mainly in adults.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

The source of immune cells that are pre-activated and expanded may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

In one exemplary method, the NK cells may be derived from a biological sample, such as one or more cord blood units. The cord blood unit may be a frozen cord blood unit. The cord blood unit may be thawed and subjected to a ficoll gradient to obtain mononuclear cells. The mononuclear cells may be depleted of CD3, CD14, and CD19 positive cells, such as by CliniMAS. The negatively-selected NK cells may then be cultured in the presence of APCs and IL-2 (e.g., 200 U/mL). The NK cells may then be transduced with retroviral supernatant expressing a CAR and cultured with γ-irradiated APCs and IL-2. Finally, the cells may be sorted for positive expression of the CAR, such as CD56, CD16, CD3, CD19, CD14, or CD45.

The immune cells generated according to the present methods have many potential uses, including experimental and therapeutic uses. In particular, it is envisaged that such cell populations will be extremely useful in suppressing undesirable or inappropriate immune responses. In such methods, a small number of immune cells are removed from a patient and then manipulated and expanded ex vivo before reinfusing them into the patient. Examples of diseases which may be treated in this way are autoimmune diseases and conditions in which suppressed immune activity is desirable, e.g., for allo-transplantation tolerance. A therapeutic method could comprise providing a mammal, obtaining immune cells from the mammal; expanding the immune cells ex vivo in accordance with the methods of the present methods as described above; and administering the expanded immune cells to the mammal to be treated.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In certain embodiments, the immune cells are administered in combination with a second therapeutic agent. For example, the second therapeutic agent may comprise T cells, an immunomodulatory agent, a monoclonal antibody, or a chemotherapeutic agent. In non-limiting examples, the immunomodulatory agent is lenolidomide, the monoclonal antibody is rituximab, of atumab, or lumiliximab, and the chemotherapeutic agent is fludarabine or cyclophosphamide.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

Desirably an effective amount or sufficient number of the isolated transduced immune cells is present in the composition and introduced into the subject such that long-term, specific, anti-tumor responses are established to reduce the size of a tumor or eliminate tumor growth or regrowth than would otherwise result in the absence of such treatment. Desirably, the amount of immune cells reintroduced into the subject causes a 10%, 20%, 30%, 40%, 50%, 60%, 70%, 80%, 90%, 95%, 98%, or 100% decrease in tumor size when compared to otherwise same conditions wherein the immune cells are not present.

Accordingly, the amount of immune cells administered should take into account the route of administration and should be such that a sufficient number of the immune cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of immune cells desirably should be sufficient to provide in the subject being treated at least from about 1×10⁶ to about 1×10⁹ immune cells, even more desirably, from about 1×10⁷ to about 5×10⁸ immune cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of immune cells to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### VII. Kits

In some embodiments, a kit that can include, for example, one or more media and components for the production of UAPCs and/or immune cells is provided. Such formulations may comprise a cocktail of factors, in a form suitable for combining with APCs and/or immune cells. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Combination of mbIL21, 4-1BBL, and SLAM (CD48/CS1)

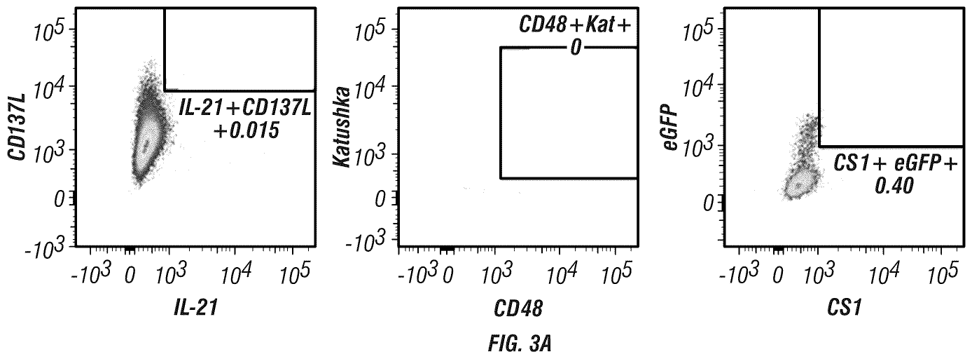

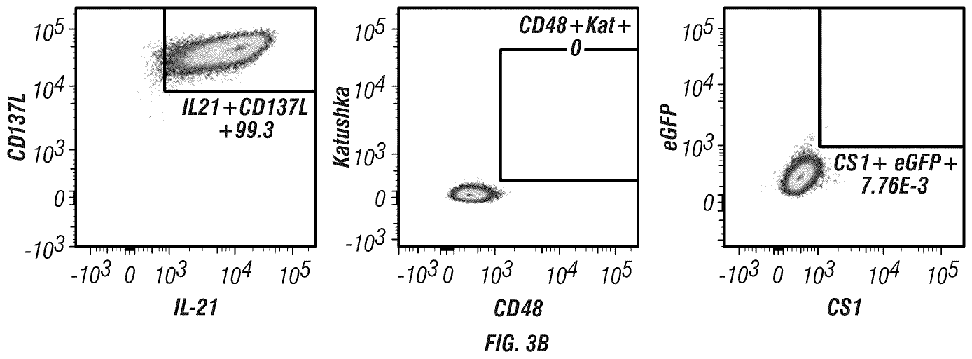

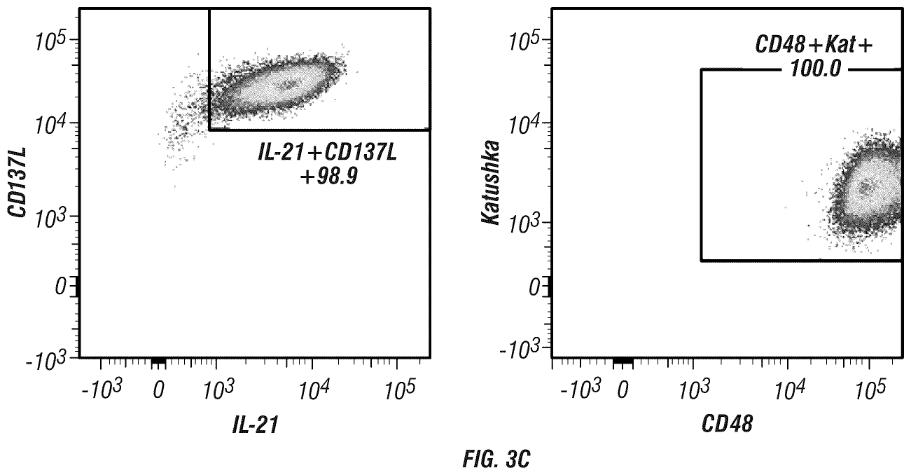

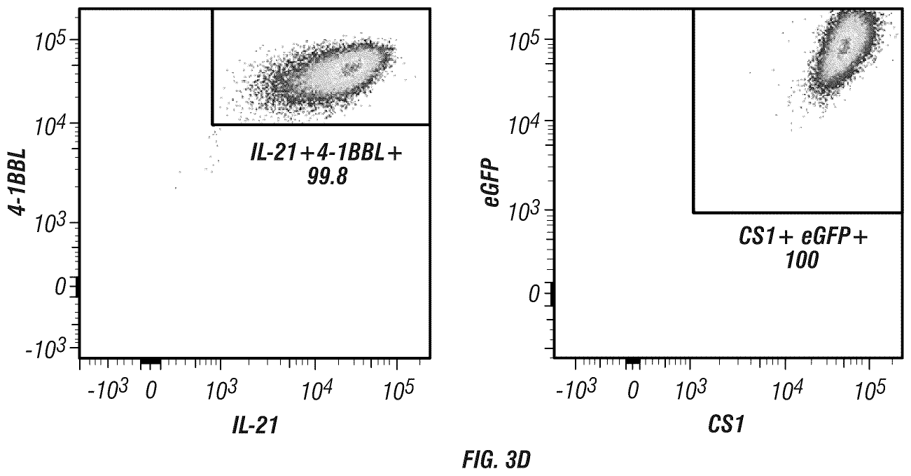

Universal antigen presenting cells (UAPCs) were generated by transduction of K562 cells with retroviral constructs for membrane-bound IL-21 and 41BBL (CD137 ligand) with either CD48-Katushka or CS1-EGFP. The MMLV-retroviral construct maps and annotations are shown in FIGS. 4A-4D. Clone 46 (FIG. 3B) was produced from enforced expression of mbIL-21 and 41BBL in NK cell-sensitive K562 (HLA-A⁻, HLA-B⁻) APCs (FIG. 3A). UAPC was generated by enforced expression of mbIL-1, 41BBL, and CD48 in the K562 cells (FIG. 3C). UAPC2 was generated by enforced expression of mbIL-21, 41BBL, and CS1 in the K562 cells (FIG. 3D).

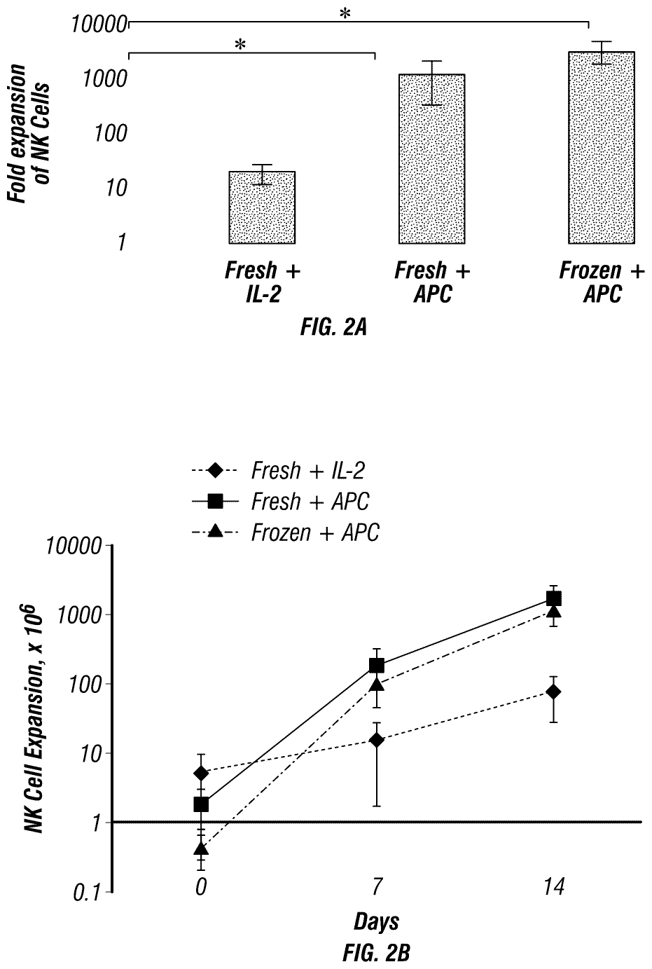

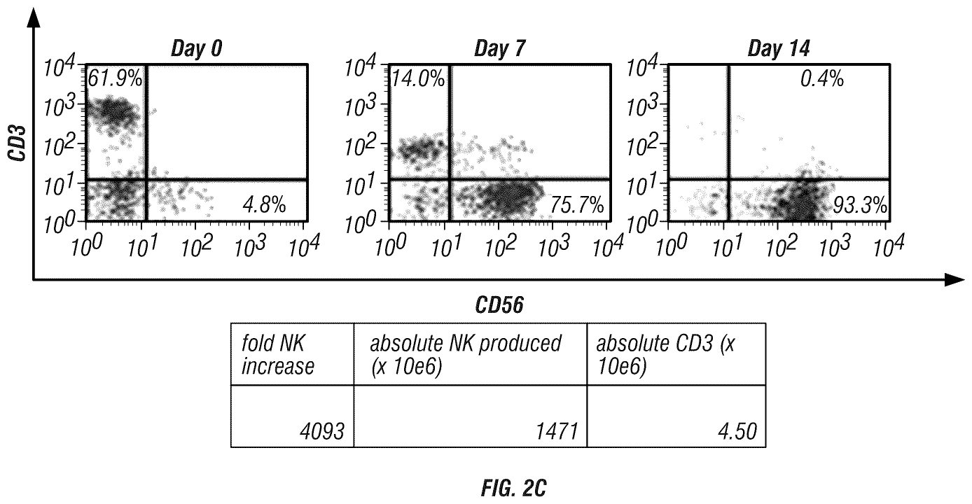

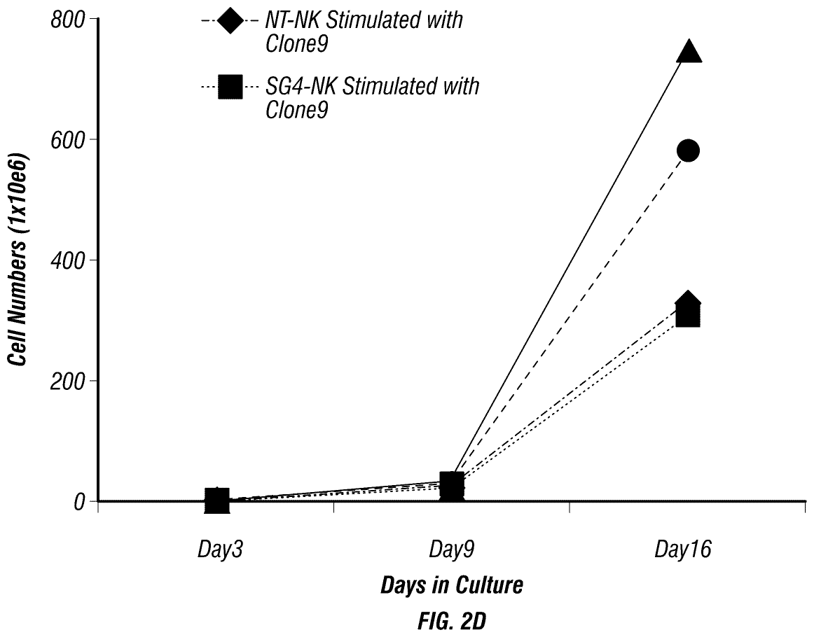

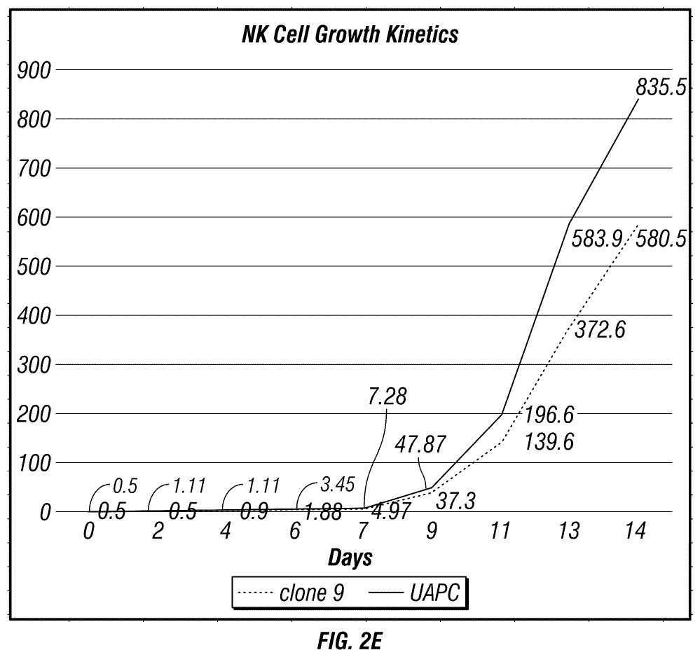

Numeric expansion of NK cells was performed with activation by clone 46, UAPC, or UAPC2. Proliferation and expansion of NK cells by APC co-culture showed that Clone 46 was superior in its ability to expand both non-transduced NK cells (NT-NK) and CAR-transduced (SG4-NK) as compared to the previously published clone 9 (FIG. 2B).

Expansion of NK cells by co-culture with UAPC was demonstrated to be superior in its ability to reduce NK cell doubling time to 31.38 hours compared to the previously published clone 9 (doubling time of 33 hours), yielding a proliferative advantage of 44% (1671-fold expansion for UAPC vs 1161-fold expansion for clone 9) over 2 weeks (FIG. 2E). The relationship between doubling time, fold expansion, and starting cell population is encapsulated by the following formula:

$${N}_{f} = {N}_{b} {e}^{\frac{T}{{T}_{D}} ln 2}$$

where N_{f} is the final cell number, N_{b} is the starting cell, e=2.71828 (mathematical constant also known as Euler's number), T is the time span of culture in hours, T_{D} is the doubling time in hours. The doubling time affects the final cell number in an exponential manner, thus compounding the proliferative advantage over the designated cell culture period, enabling clinically relevant numbers of cells to be harvested in a compacted timeframe. Therefore, the present methods allow for the efficient production of NK cells by co-culture with the UAPCs.

The present platform technology takes advantage of specific combinatorial therapeutic pathways to provide practical methods for the manufacture of large numbers of clinical-grade highly functional and cytotoxic NK cells intended for cancer immunotherapy. The expanded cells are 100% NK cells, with no detectable T cells, avoiding unintended graft-versus-host side effects common in many immune cell preparations.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Czerkinsky et al., J. Immunol. Methods 1988; 110:29-36.
Fast et al., Transfusion 2004; 44:282-5.
He Y, et al. Journal of immunology research. 2014; 2014:7.
International Publication No. PCT/US95/01570
International Publication No. WO2000/06588
International Publication No. WO2005/035570
Olsson et al. J. Clin. Invest. 1990; 86:981-985.
Taitano et al., The Journal of Immunology, 196, 2016.
U.S. Pat. No. 5,939,281
U.S. Pat. No. 6,218,132
U.S. Pat. No. 6,264,951
U.S. Pat. No. 7,488,490
U.S. Patent Publication No. 2007/0078113

What is claimed is:

1. A universal antigen presenting cell (UAPC) engineered to express (1) CD48 and/or CS1 (CD319), (2) membrane-bound interleukin-21 (mbIL-21), and (3) 41BB ligand (41BBL).

2. The UAPC of claim 1, wherein the UAPC expresses CD48.

3. The UAPC of claim 1, wherein the UAPC expresses CS1.

4. The UAPC of claim 1, wherein the UAPC expresses CD48 and CS1.

5. The UAPC of claim 1, wherein the UAPC has essentially no expression of endogenous HLA class I, II, or CD1d molecules.

6. The UAPC of claim 1, wherein the UAPC expresses ICAM-1 (CD54) and LFA-3 (CD58).

7. The UAPC of claim 1, wherein the UAPC is a leukemia cell-derived UAPC.

8. The UAPC of claim 7, wherein the leukemia-cell derived UAPC is further defined as a K562 cell.

9. The UAPC of claim 1, wherein the UAPC has been engineered by retroviral transduction.

10. The UAPC of claim 9, wherein the retroviral transduction is further defined as transduction using a viral construct of SEQ ID NO: 1 and/or SEQ ID NO:2.

11. The UAPC of any one of claims 1-10, wherein the UAPC is irradiated.

12. A method for expanding immune cells comprising culturing the immune cells in the presence of an effective amount of UAPCs of any one of claims 1-11.

13. The method of claim 12, wherein the immune cells and UAPCs are cultured at a ratio of 3:1 to 1:3.

14. The method of claim 12, wherein the immune cells and UAPCs are cultured at a ratio of 1:2.

15. The method of claim 12, wherein the expanding is in the presence of IL-2.

16. The method of claim 15, wherein the IL-2 is present at a concentration of 10-500 U/mL.

17. The method of claim 15, wherein the IL-2 is present at a concentration of 100-300 U/mL.

18. The method of claim 15, wherein the IL-2 is present at a concentration of 200 U/mL.

19. The method of claim 15, wherein the IL-2 is recombinant human IL-2.

20. The method of claim 15, wherein the IL-2 is replenished every 2-3 days.

21. The method of claim 12, wherein the UAPCs are added at least a second time.

22. The method of claim 12, wherein the immune cells are NK cells or T cells.

23. The method of claim 12, wherein the immune cells are NK cells.

24. The method of claim 12, wherein the immune cells are T cells.

25. The method of claim 12, wherein the immune cells are derived from cord blood (CB), peripheral blood (PB), stem cells, or bone marrow.

26. The method of claim 12, wherein the stem cells are induced pluripotent stem cells.

27. The method of claim 12, wherein the immune cells are obtained from CB.

28. The method of claim 27, wherein the CB is pooled from 2 or more individual cord blood units.

29. The method of claim 27, wherein the CB is pooled from 3, 4, 5, 6, 7, or 8 individual cord blood units.

30. The method of claim 23, wherein the NK cells are CB mononuclear cells (CBMCs).

31. The method of claim 23, wherein NK cells are further defined as CD56+NK cells.

32. The method of claim 12, wherein the method is performed in serum-free media.

33. The method of any one of claims 12-32, wherein the immune cells are engineered to express a chimeric antigen receptor (CAR).

34. The method of claim 33, wherein the CAR comprises a humanized antigen-binding domain.

35. The method of claim 33, wherein the CAR comprises a CD19, CD123, mesothelin, CD5, CD47, CLL-1, CD33, CD99, U5snRNP200, CD200, CS1, BAFF-R, ROR-1, or BCMA antigen-binding domain.

36. The method of claim 33, wherein the CAR comprises a CD19 or CD123 antigen-binding domain.

37. The method of claim 33, wherein the CAR comprises IL-15.

38. The method of claim 33, wherein the CAR comprises a suicide gene.

39. The method of claim 38, wherein the suicide gene is CD20, CD52, EGFRv3, or inducible caspase 9.

40. A population of expanded immune cells produced according to the methods of any one of claims 12-39.

41. A pharmaceutical composition comprising the population of expanded immune cells of claim 40 and a pharmaceutically acceptable carrier.

42. A composition comprising an effective amount of the expanded immune cells of claim 40 for use in the treatment of a disease or disorder in a subject.

43. A method of treating a disease or disorder in a subject comprising administering a therapeutically effective amount of the expanded immune cells of claim 40 to the subject.

44. The method of claim 43, wherein the disease or disorder is cancer, inflammation, graft versus host disease, transplant rejection, an autoimmune disorder, an immunodeficiency disease, a B cell malignancy, or an infection.

45. The method of claim 44, wherein the cancer is a leukemia.

46. The method of claim 45, wherein the leukemia is an acute lymphoblastic leukemia (ALL), chronic lymphocytic leukemia (CLL), acute myelogenous leukemia (AML), or a chronic myelogenous leukemia (CML).

47. The method of claim 43, wherein the immune cells are allogeneic.

48. The method of claim 43, wherein the immune cells are autologous.

49. The method of claim 43, wherein the immune cells are NK cells or T cells.

50. The method of claim 43, wherein the immune cells are NK cells.

51. The method of claim 43, wherein the disorder is graft versus host disease (GVHD).

52. The method of claim 43, wherein the disorder is multiple sclerosis, inflammatory bowel disease, rheumatoid arthritis, type I diabetes, systemic lupus erythrematosus, contact hypersensitivity, asthma or Sjogren's syndrome.

53. The method of claim 43, wherein the subject is a human.

54. The method of claim 43, further comprising administering at least a second therapeutic agent.

55. The method of claim 54, wherein the at least a second therapeutic agent is a therapeutically effective amount of an anti-cancer agent, immunomodulatory agent, or an immunosuppressive agent.

56. The method of claim 55, wherein the anti-cancer agent is chemotherapy, radiotherapy, gene therapy, surgery, hormonal therapy, anti-angiogenic therapy or immunotherapy.

57. The method of claim 55, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.

58. The method of claim 54, wherein immune cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

59. The method of claim 54, wherein the second therapeutic agent is an antibody.

60. The method of claim 59, wherein the antibody if a monoclonal, bispecific, or trispecific antibody.

61. The method of claim 60, wherein the antibody is rituximab.


SEQUENCE LISTING (2 sequences)

SEQ ID NO: 1 - DNA, 8202 bp, synthetic construct
  misc_feature 1..8202: note = CS1 Construct
  source 1..8202: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgcgactgattttatgcgcctgcgtcggtactagttagctaactagctctgtatctggcggacccgtgggctggaactgacgagttcggaacacccggccgcaaccctgggagacgtcccagggacttcgggggccggctttttgtggcccgacctgagtcctaaaatcccgatcgtttaggactctttggtgcaccccccttaggcaggagggatatgtggttctggtaggagacgagaacctaaaacagttcccgcctccgtctgaatttttgctttcggtttgggaccgaagccgcgccgcgcgtcttgtctgctgcagcatcgttctgtgttgtcgctctgtctgactgtgtttctgtatttgtctgaaaatatgggcccgggctagcctgttaccactccctgctaagtttgaccttaggtcactggaaagatgtcgagcggatcgctcacaaccagtcggtagatgtcagcagaagagacgttgggttaccttctgctctgcagaatggccaacctttaacgtcggatggccgcgaggcacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttggcaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacggcaagtctggagacctctggcggcagcctaccaagaacaactggaccgaccggtggtacctcacccttgcaccgagtcggcgacacagtgtgggtccgccgacaccagactaagaacctagaacctcgctggaaaggcgaccttacacagtcctgctgaccacccccaccgccctcaaagtagacggcatcgcagcttggatacacgccgcccacgtgaaggctgccgaccccgggggtggaccatcctctagactgccatgctcgaggccggcagccccacctgcctgaccctgatctacatcctgtggcagctgaccggcagcgccgccagcggccccgtgaaggagctggtgggcagcgtgggcggcgccgtgaccttccccctgaagagcaaggtgaagcaggtggacagcatcgtgtggaccttcaacaccacccccctggtgaccatccagcccgagggcggcaccatcatcgtgacccagaacagaaacagagagagagtggacttccccgacggcggctacagcctgaagctgagcaagctgaagaagaacgacagcggcatctactacgtgggcatctacagcagcagcctgcagcagcccagcacccaggagtacgtgctgcacgtgtacgagcacctgagcaagcccaaggtgaccatgggcctgcagagcaacaagaacggcacctgcgtgaccaacctgacctgctgcatggagcacggcgaggaggacgtgatctacacctggaaggccctgggccaggccgccaacgagagccacaacggcagcatcctgcccatcagctggagatggggcgagagcgacatgaccttcatctgcgtggccagaaaccccgtgagcagaaacttcagcagccccatcctggccagaaagctgtgcgagggcgccgccgacgaccccgacagcagcatggtgctgctgtgcctgctgctggtgcccctgctgctgagcctgttcgtgctgggcctgttcctgtggttcctgaagagagagagacaggaggagtacatcgaggagaagaagagagtggacatctgcagagagacccccaacatctgcccccacagcggcgagaacaccgagtacgacaccatcccccacaccaacagaaccatcctgaaggaggaccccgccaacaccgtgtacagcaccgtggagatccccaagaagatggagaacccccacagcctgctgaccatgcccgacacccccagactgttcgcctacgagaacgtgatcggcagcacctccggcagcggcgcgcctggcagcggcgagggcagcaccaagggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaagctttttttttaaagatcctacactatagttcaagctagactattagctactctgtaacccagggtgagcccttgaagtcatgggtagcctgctgttttagccttcccacatctaagattacaggtatgagctatcgcatttttggtatattgattgattgattgattgatgtgtgtgtgtgtgattgtgtttgtgtgtgtgacgctgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtggctgtgtgtgtgtgtgtgtgtgtgtgttgtgaaaaaatattctatggtagtgagagccaacgctccgggcctcaggtgtcaggttggtttttgagacagagtctttcacttagcttggaattcactggccgtcgttgcttacaacgtcgtgactgggaaaaccctggcgttacccaacttaatcgccttgcagcacatccccctgcttcgccagctggcgtaatagcgaagaggcccgcaccgatcgcccttcccaacagttgcgcagcctggcaatggcgaatggcgcctgatgcggtattttctccttacgcatctgtgcggtatttcacaccgcatagctggtgcactctcagtacaatctgctctgatgccgcatagttaagccagccccgacacccgccaacagccccgctgacgcgccctgacgggcttgtctgctcccggcatccgcttacagacaagctgtgaccgtcgctccgggagctgcatgtgtcagaggttttcaccgtcatcaccgaaacgcgcgagacgaaagggcctcgcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactgccattaggcaccccaggctttacactttatgcttccggctcgtatgttgtgtggaattgtgagcggagctaacaatttcacacaggaaacagctatgaccatgattacgccaagctttgctcttaggagtttcctgcaatacatcccaaactcaaatatataaagcatttgacttgttctatgccctagggggcggggggaaggcctaagccagctttttttaacatttaaaatgttaattccattttaaatgcacagatgtttttatttcgcataagggtttcaatgtgcatgaatgctgcaatattcctgttaccaaagctagtataaataaaaatagataaacgtggaaattacttagagtttctgtcattaacgtttccttcctcagttgacaacataaatgcgcgctgctgagcaagccagtttgcatctgtcaggatcaatttcccattatgccagtcatattaattgcactagtcaattagttgatttttatttttgacatatacatgtg

SEQ ID NO: 2 - DNA, 7935 bp, synthetic construct
  misc_feature 1..7935: note = CD48 Construct
  source 1..7935: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatcgactgattttatgcgcctgcgtcggtactagttagctaactagctctgtatctggcggacccgtggctggaactgacgagttcggaacacccggccgcaaccctgggagacgtcccagggacttcgggggccgctttttgtggcccgacctgagtcctaaaatcccgatcgtttaggactctttggtgcaccccccttagcaggagggatatgtggttctggtaggagacgagaacctaaaacagttcccgcctccgtctgaatttttgctttcggtttgggaccgaagccgcgccgcgcgtcttgtctgctgcagcatcgttctgtgttgtcctctgtctgactgtgtttctgtatttgtctgaaaatatgggcccgggctagcctgttaccactccctctaagtttgaccttaggtcactggaaagatgtcgagcggatcgctcacaaccagtcggtagatgtcacagaagagacgttgggttaccttctgctctgcagaatggccaacctttaacgtcggatggccgcgagcacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgcaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgcaagtctggagacctctggcggcagcctaccaagaacaactggaccgaccggtggtacctcacccttcaccgagtcggcgacacagtgtgggtccgccgacaccagactaagaacctagaacctcgctggaaagcgaccttacacagtcctgctgaccacccccaccgccctcaaagtagacggcatcgcagcttggatacacgccgcccacgtgaaggctgccgaccccgggggtggaccatcctctagactgccatgctcgaggtgggtgaggatagcgtgctgatcaccgagaacatgcacatgaaactgtacatggagggcaccgtgaacgaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaaacctttatcaaccacacccagggcatccccgacttctttaagcagtccttccctgagggcttcacatgggagaggatcaccacatacgaagacgggggcgtgctgaccgctacccaggacaccagcctccagaacggctgcctcatctacaacgtcaagatcaacggggtgaacttcccatccaacggccctgtgatgcagaagaaaacactcggctgggaggccagcaccgagatgctgtaccccgctgacagcggcctgagaggccatgcccagatggccctgaagctcgtgggcgggggctacctgcactgctccctcaagaccacatacagatccaagaaacccgctaagaacctcaagatgcccggcttctacttcgtggaccggagactggaaagaatcaaggaggccgacaaagagacctacgtcgagcagcacgagatggctgtggccagatactgcgacctgcctagcaaactggggcacagcggcagcacctccggcagcggcgcgcctggcagcggcgagggcagcaccaagggcgagggcaggggaagtcttctaacatgcggggacgtggaggaaaatcccgggcccatgtgcagcagaggctgggacagctgcctggccctggagctgctgctgctgcccctgagcctgctggtgaccagcatccagggccacctggtgcacatgaccgtggtgagcggcagcaacgtgaccctgaacatcagcgagagcctgcccgagaactacaagcagctgacctggttctacaccttcgaccagaagatcgtggagtgggacagcagaaagagcaagtacttcgagagcaagttcaagggcagagtgagactggacccccagagcggcgccctgtacatcagcaaggtgcagaaggaggacaacagcacctacatcatgagagtgctgaagaagaccggcaacgagcaggagtggaagatcaagctgcaggtgctggaccccgtgcccaagcccgtgatcaagatcgagaagatcgaggacatggacgacaactgctacctgaagctgagctgcgtgatccccggcgagagcgtgaactacacctggtacggcgacaagagacccttccccaaggagctgcagaacagcgtgctggagaccaccctgatgccccacaactacagcagatgctacacctgccaggtgagcaacagcgtgagcagcaagaacggcaccgtgtgcctgagccccccctgcaccctggccagaagcttcggcgtggagtggatcgccagctggctggtggtgaccgtgcccaccatcctgggcctgctgctgacctgacgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgttctgtttgtttgtttgttggttggttggttaatttttttttaaagatcctacactatagttcaagctagactattagctactctgtaacccagggtgaccttgaagtcatgggtagcctgctgttttagccttccccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgctatgtatgtgtgtgtgtgagtgtgtgtgtgtgtgtgtgcatgtgtgtgtgtgtgactgtgtctatgctgtatgactgtgtgtgtgtgtgtgtgtgtgtgtgtgtgtgtgtgtgtgtgtgtgttgtgaaaaaatcattctatggtagtgagagccaacgctccggctcaggtgtcaggttggtttttgagacagagtctttccacttagcttggaattcactggccgtcgttttacaacgtcgtgactgggaaaaccctggcgttacccaacttaatcgccttgcagcacatccccctttcgccagctggcgtaatagcgaagaggcccgcacccgatcgcccttcccaacagttgcgcagcctgaatggcgaatggcgcctgatgcggtattttctccttcacgcatctgtgcggtatttcacaccgcatatggtgcactctcagtacaatctgctctgatgccgcatagttaagccagccccgacacccgccaacacccgctgacgcgccctgacgggcttgtctgctcccgcgcatccgcttacagacaagctgtgaccgtctccgggagctgcatgtgtcagaggttttcaccgtcatcaccgaaacgcgcgagacgaaagggcctcgtgatacgcctatttttataggttaatgtcatgatacataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatccccctgattctgtggataaccgtattaccgcctttgagtgagctgataccgctcgccgcagccgaacgcaccgagcgcagcgagtcagtgagcgaggaagcggaagagcgcccaatacgcaaaccgcctctcccccgcgcgttggccgattcattaatgcagctggcacgacaggtttcccgactggaaagcgggcagtgagcgcaacgcaattaatgtgagttagctcactcattaggcaccccaggctttacactttatgcttccgcgctcgtatgttgtgtggaattgtgagcggataacaatttcacacaggaaacagctatgaccatgatctacgccaagctttgctcttaggagtttcctaatacatcccaaactcaaatatataaagcatttgacttgttctatgccctagggggcggggggaagctaagccagctttttttaacatttaaaatgttaattcccattttaaatgcacagatgtttttatttcataagggtttcaatgtgcatgaatgctgcaatattccctgttaccaaagctagtataaataaaaatagataaacgtggaaattacttagagtttctgtcattacacgtttccttcctcagttgacaacataaatgcgctgctgagcaagccagtttgcatctgtcaggatccaatttcccattatgccagtcatattaattactagtcaattagttgatttttatttttgacatatacatgtg